(19) United States
(12) Patent Application Publication
(10) Pub. No.: US 2016/0256139 A1
(43) Pub. Date: Sep. 8, 2016

(54) BIOPSY SAMPLE COLLECTION DEVICE

(71) Applicant: Merit Medical Systems, Inc., South Jordan, UT (US)

(72) Inventors: Daniel Wayne Hadley, Herriman, UT (US); Aaron Hopkinson, Herriman, UT (US); Barton P. Gill, South Jordan, UT (US); Chad Sievers Carson, Riverton, UT (US)

(21) Appl. No.: 15/058,536

(22) Filed: Mar. 2, 2016

Related U.S. Application Data
(60) Provisional application No. 62/128,317, filed on Mar. 4, 2015.

(51) Int. Cl. A61B 10/04; A61B 1/267

(52) U.S. Cl. CPC A61B 10/04; A61B 1/2676

(57) ABSTRACT

Biological sample collection devices are disclosed that resist disorientation and tipping to remain in an upright orientation. A biological sample collection device can be a lid that couples to a sample collection jar to enable coupling to a scope device and a suction source. A biological sample collection device can include a suction port to couple to the suction source and a scope port to couple to the scope device. The scope port can include an angled connection member that angles in line and away from the suction port. The biological sample collection device can also include a suction hose clip and/or a light pipe clip to secure the biological sample collection device relative to the suction hose and/or a light pipe. A biological sample collection device can also couple to a traditional sample collection jar and include a suction hose clip and/or a light pipe clip.

### RELATED APPLICATIONS

This application claims priority to U.S. Provisional Application No. 62/128,317, filed on Mar. 4, 2015 and titled “Biopsy Sample Collection Device” which is hereby incorporated by reference in its entirety.

### TECHNICAL FIELD

The present disclosure relates generally to devices for collecting a biological sample, such as collecting a biological sample for biopsy.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is an isometric view of a biopsy sample collection device, according to one embodiment.

FIG. 2 is a top perspective view of the biopsy sample collection device of FIG. 1.

FIG. 3 is a bottom perspective view of the biopsy sample collection device of FIG. 1.

FIG. 4 is a top view of the biopsy sample collection device of FIG. 1.

FIG. 5 is a bottom view of the biopsy sample collection device of FIG. 1.

FIG. 6 is a side view of the biopsy sample collection device of FIG. 1.

FIG. 7 is a side cross sectional view of the biopsy sample collection device of FIG. 1.

FIG. 8 is a perspective view of a biopsy sample collection device, according to another embodiment, uncoupled from a biopsy collection jar.

FIG. 9 is a perspective view of the biopsy sample collection device of FIG. 8 coupled to the biopsy collection jar.

FIG. 10 is a perspective view of a biopsy sample collection device, according to still another embodiment, coupled to a biopsy collection jar.

FIG. 11 is a side perspective view of a biopsy sample collection device, according to still another embodiment.

### DETAILED DESCRIPTION

Scope devices have enabled an increasing number of minimally invasive medical procedures. Scope devices are used to gain views of numerous body cavities (e.g., organs, lumens, and the like) that may be otherwise inaccessible absent an invasive surgery.

Advances in scope devices have enabled additional medical procedures beyond merely viewing body cavities. Tissue and other samples can be collected for biopsy. Tissue samples may be cut and suctioned through a suction lumen of a scope device for collection. Mucous and other fluid or fluid-like samples can be suctioned through the suction lumen of a scope device. Scope devices may also include a lavage lumen to deposit saline or other fluid into a viewed body cavity, and the saline or fluid is then suctioned to collect a biological sample. A collected biological sample can be a biopsy sample that is examined to provide a practitioner with important insight and information as to the health and condition of a body cavity.

Minimally invasive access to and treatment of the airway has been enabled by advances in scope devices. Bronchoscopy is a procedure that visualizes the tracheobronchial tree (airway) by placing a scope instrument inside the airway. A bronchoscope inserted into the airway may include suction for obtaining biological samples of tissue and/or fluid for culturing and/or biopsy. As used herein, biological sample includes any tissue, fluid, and/or other material removed from a living body, and may be a biopsy sample. As used herein, biopsy includes examination of any tissue, fluid, and/or other material removed from a living body to discover the presence, cause, or extent of a disease.

Bronchoalveolar lavage (BAL) is one example of an effective procedure for retrieval of biopsy samples from the airway. A bronchoscope is passed through the mouth or nose into the lungs and a saline solution or other appropriate fluid is squirted into a small portion of the airway or a lung and then collected for examination. BAL typically may be performed to diagnose lung disease. BAL is a common manner to sample the components of the epithelial lining fluid (ELF) and to determine the protein composition of the pulmonary airways, and it is often used in immunological research as a means of sampling cells or pathogen levels in the lung.

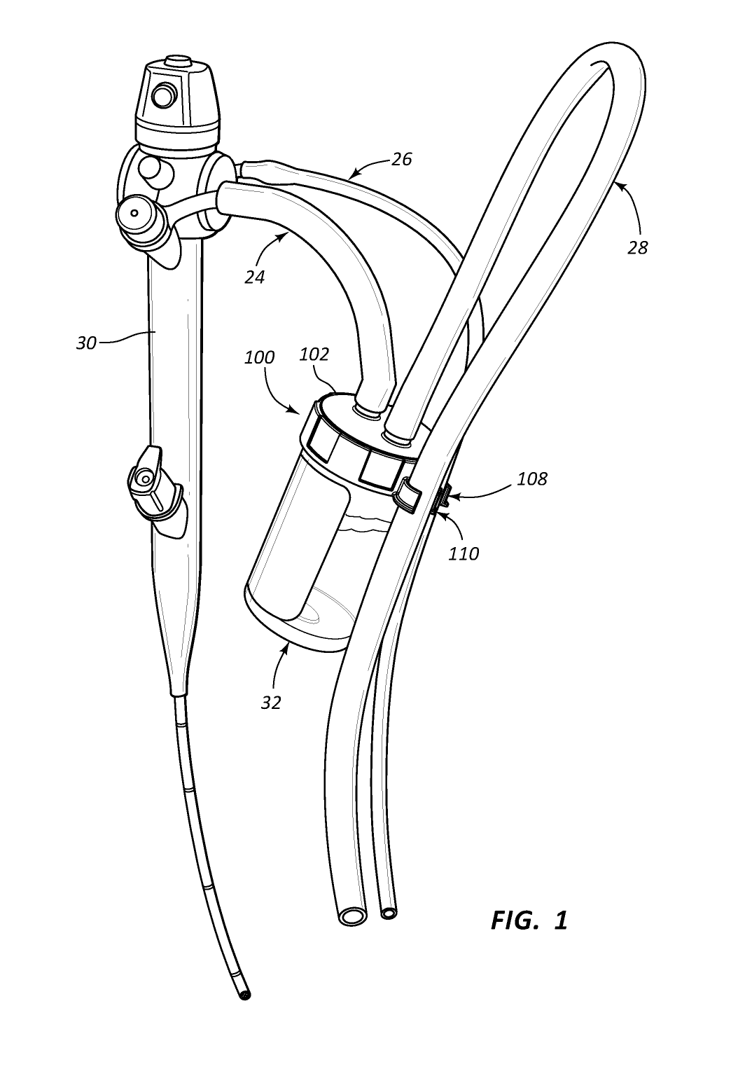

Biological samples, such as biopsy samples, can be collected during a scope procedure by providing a suction source to a suction lumen of the scope device. A scope device that has a suction lumen is convenient for a practitioner to collect biopsy samples because a suction port is disposed near the optic element of the scope. A sample collection jar can be positioned along a suction line, coupled to and disposed between the scope device and the suction source, as shown in FIG. 1 and discussed below with reference to the same. The sample collection jar may be configured to trap or otherwise retain a biopsy sample suctioned through a suction lumen of the scope device. The sample collection jar may include or be coupled to a lid that includes a suction source port and a scope port. The suction source port is to couple to a suction source and to provide fluid communication to an interior of the sample collection jar, such that the suction source generates a vacuum (or otherwise transfers or creates a negative pressure) within the sample collection jar. The scope port also provides fluid communication to the interior of the sample collection jar to transfer negative pressure within the sample collection jar to the scope device. The scope port receives samples from the scope device that are suctioned through the suction lumen of the scope device.

Generally, a sample collection jar can collect and safely retain a biological sample, so long as the sample collection jar remains in an upright orientation. However, tipping of the sample collection jar can allow a collected biological sample to shift near to the suction source port within the jar and to be inadvertently suctioned out of the jar. A biological sample suctioned out of the sample collection jar, such as due to tipping of the jar, is lost, which may defeat the purpose of the scope procedure.

To collect a biological sample during a scope procedure, such as a BAL procedure, can present challenges in maintaining the sample collection jar in an upright orientation. Typically, manipulation of the scope device, as is common during scope procedures, can inadvertently cause the sample collection jar to tip. In other words, manipulation of the scope device can result in inadvertent tipping or even inverting of the sample jar. A practitioner, while performing a scope procedure to collect a biological sample, may twist and turn and otherwise manipulate the scope device to obtain desired access and/or views into the target body cavity, and such manipulation of the scope device can result in altering the orientation of the biopsy sample jar coupled to the scope device. Tipping of the biological sample jar can cause a collected sample within the jar to shift, flow, or otherwise move near to a suction source port. When collected samples move near to the suction port, there is a high risk of loss of the collected samples, through the suction source port and into the suction source.

A biological sample collection device that resists disorientation and tipping to remain in an upright (or substantially upright) orientation may be desirable. The present disclosure is directed to embodiments of biological sample collection devices that attempt to address the foregoing and other shortcomings of presently available biopsy sample collection jars.

It will be readily understood with the aid of the present disclosure that the components of the embodiments, as generally described and illustrated in the figures herein, could be arranged and designed in a variety of configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The terms “proximal” and “distal” refer to opposite ends of a medical device. As used herein, the proximal end of a medical device is the end nearest to a practitioner during use, while the distal end is the opposite end. For example, the proximal end of a stent refers to the end nearest to the practitioner when the stent is disposed within, or being deployed from, a deployment device. For consistency throughout, these terms remain constant in the case of a deployed stent, regardless of the orientation of the stent within the body. In the case of an esophageal stent—deployed through the mouth of a patient—the proximal end will be nearer to the head of the patient and the distal end nearer to the stomach when the stent is in a deployed position.

FIG. 1 is an isometric view of a biopsy sample collection device 100, according to one embodiment. The biopsy sample collection device 100 may be a lid that couples to a sample collection jar 32 and enables coupling of the sample collection jar 32 to a scope device 30 and a suction source or generator (not shown). More specifically, the biopsy sample collection device 100 couples to a sample collection tube 24 in fluid communication with a collection lumen of the scope device 30 and the biopsy sample collection device 100 couples to a suction hose 28 from the suction source. The biopsy sample collection device 100 facilitates maintaining an upright orientation of the sample collection jar 32 during a biopsy collection procedure using the scope device 30 by enabling securement of the biopsy sample collection device 100 relative to one or more of the suction hose 28 and a light pipe 26 of the scope device 30. Stated differently, a securement of the biopsy sample collection device 100 relative to one or more of the suction hose 28 and the light pipe 26 can facilitate maintaining the sample collection jar 32 in an upright orientation. The scope device 30 may be an endoscope used for visual examination of an interior of a body lumen or body cavity (e.g., a hollow organ such as the colon, esophagus, trachea, lungs, stomach). The biopsy sample collection device 100 includes a lid body 102, a suction hose clip 110, and a light pipe clip 108. The biopsy sample collection device 100 may be coupled to the sample collection jar 32, such as by complementing threads on the biopsy sample collection device 100 and the jar 32.

The sample collection tube 24 couples to the biopsy sample collection device 100 and facilitates communication of samples from the scope device 30 to the biopsy sample collection device 100. The sample collection tube 24 may be formed of a polymer material, such as Tygon® tubing or silicon tubing, that is both flexible and also sufficiently rigid to resist kinking and to position the biopsy sample collection device 100 so as to maintain the sample collection jar 32 in an upright orientation.

The light pipe 26 provides a light source for the scope device 30. The light pipe clip 108 of the biopsy sample collection device 100 may fasten to or otherwise engage the light pipe 26 to secure the biopsy sample collection device 100 relative to the light pipe 26 to maintain the sample collection jar 32 in an upright orientation. Described differently, the light pipe clip 108 secures the biopsy sample collection device 100 to the light pipe 26 to maintain an upright orientation of the sample collection jar 32. The light pipe clip 108 may be disposed on the lid body 102. For example, the light pipe 26 may be coupled at a lateral edge of the lid body 102.

The suction hose 28 couples to the biopsy sample collection device 100 and transfers a vacuum or negative pressure of a vacuum source to an internal chamber of the sample collection jar 32. The suction hose 28 can be secured or otherwise coupled to the biopsy sample collection device 100 by the suction hose clip 110. Stated differently, the suction hose clip 110 engages the suction hose 28 to secure the biopsy sample collection device 100 relative to the suction hose 28 to maintain the biopsy sample collection device 100 in an upright orientation. The suction hose 28 couples at one end to the biopsy sample collection device 100, the suction hose 28 may be looped or doubled over, and the suction hose clip 110 of the biopsy sample collection device 100 is configured to engage a doubled-over portion of the suction hose 28, as shown in FIG. 1. The suction hose 28 may be formed of a polymer material, such as Tygon® tubing or silicon tubing. The suction hose clip may be disposed adjacent the light pipe clip 108, on the distal end and the lateral edge of the lid body 102. In other embodiments, the biopsy sample collection device 100 may also include a sample trap or small basket disposed within the interior of the sample collection jar 32. The sample trap or basket may help to collect samples as they enter the sample collection jar 32.

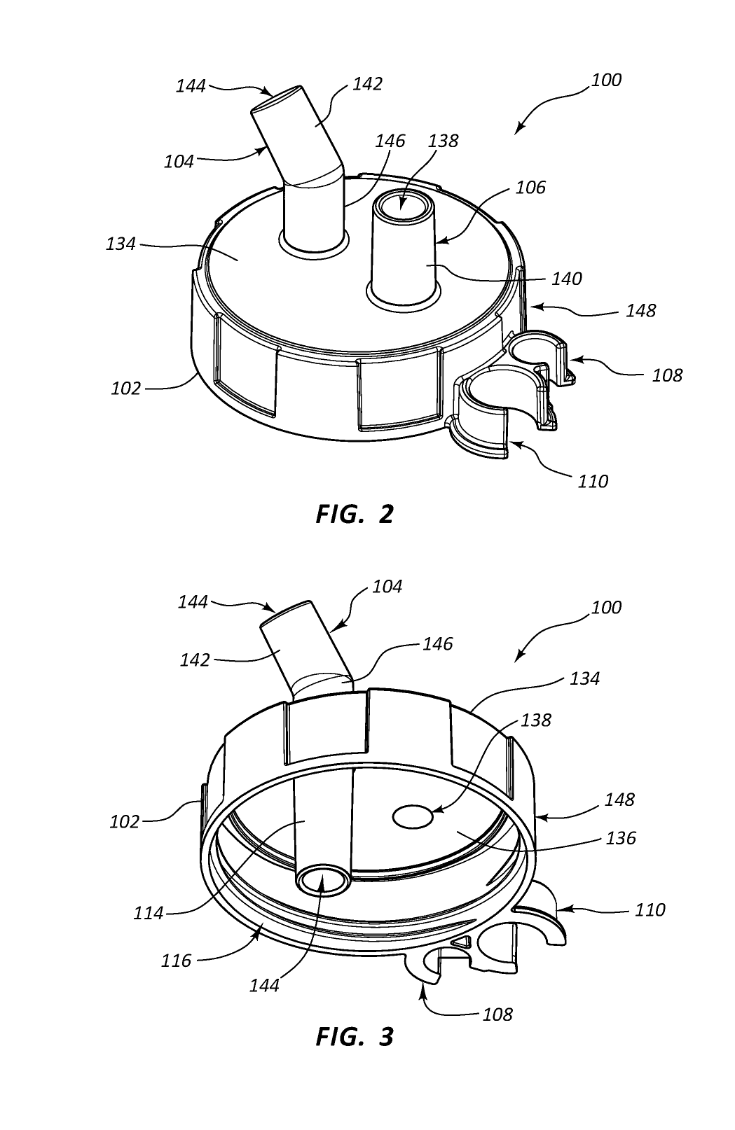

FIGS. 2 and 3 are perspective views of the biopsy sample collection device 100 of FIG. 1. Referring generally and collectively to FIGS. 2 and 3, the biopsy sample collection device 100 includes the lid body 102, the suction hose clip 110, the light pipe clip 108, and a scope port 104 and a suction source port 106, which are disposed on the lid body 102.

The suction source port 106 may include a suction source aperture 138 defined in and through the lid body 102 and a connection member 140. The suction source aperture 138 provides fluid communication from a suction source to an interior of a sample collection jar (e.g., see the jar 32 of FIG. 1) to which the biopsy sample collection device 100 may be secured. The connection member 140 extends or lengthens the suction source aperture 138 as a lumen through the suction source port 106. The connection member 140 may be configured to couple to a suction source to facilitate transfer of suction from a suction source through the suction source aperture 138. More specifically, the connection member 140 is configured to receive or otherwise couple to a suction hose 28 (see FIG. 1), which is coupled to the suction source. The suction hose 28 may couple to the connection member 140, for example, with a friction fit. The transfer of suction through the suction source aperture 138 may then create a negative pressure in the interior of the sample collection jar. The negative pressure within the jar may aid in communicating samples from a scope device to the jar, as will be described in greater detail below. In the embodiment of FIGS. 2-3, the connection member 140 of the suction source port 106 extends away from and substantially orthogonal to a top outer surface 134 of the lid body 102. In other embodiments, the suction source port 106 may couple to a suction source in a variety of different ways. For example, the suction source port 106 may be configured to couple to a suction source with a threaded socket, a lock, a clamp, a latch, or other fastening mechanism. The suction source port 106 may include one or more of these examples in conjunction with the connection member 140 or in place of the connection member 140.

The suction source aperture 138, in the illustrated embodiment of FIG. 3, is disposed in the lid body 102 substantially at an bottom inner surface 136 of the lid body 102. As can be appreciated, in other embodiments, the suction source aperture 138 may be extended or otherwise displaced from the bottom inner surface 136.

The scope port 104 of the biopsy sample collection device 100 may include an intake aperture 144 (e.g., a lumen), an upper extension member 146, an angled connection member 142, and a lower extension member 114. The intake aperture 144 may be defined in and through the lid body 102. The intake aperture 144 is extended or lengthened as a lumen through the scope port 104 (e.g., through the lower extension member 114, the upper extension member 146, and the angled connection member 142). The intake aperture 144 provides fluid communication to the interior of the sample collection jar, to transfer negative pressure from within the sample collection jar to a scope device, and to receive samples from a scope device. The transfer of negative pressure from the interior of the sample collection jar to a scope device may aid to communicate samples from a scope device to pass through the intake aperture 144 and enter the sample collection jar. In other embodiments, the intake aperture 144 may be of some size that is greater or less than the aperture size shown. The size of the intake aperture 144 in these other embodiments may facilitate the communication of fluid and samples as well as the transfer of negative pressure between the interior of the sample collection jar and the scope device.

The upper extension member 146 extends away from and substantially orthogonal to the top outer surface 134 of the lid body 102. The angled connection member 142 extends from the upper extension member 146 at an angle away from and in line with the suction source port 106. The angled connection member 142 facilitates coupling the scope port 104 to a collection lumen of the scope device. More specifically, the angled connection member 142 is configured to receive or otherwise couple to a sample collection tube 24 (see FIG. 1), which is coupled to the scope device. The sample collection tube 24 may be Tygon® tubing, silicone tubing, or other appropriately flexible yet sufficiently rigid material. The angled connection member 142 facilitates maintaining the biopsy sample collection device 100 in an upright orientation. The angled connection member 142 and the upper extension member 146 also facilitate transfer of suction from the interior of the sample collection jar through the intake aperture 144. The transfer of suction may then create a negative pressure within the collection lumen of the scope device. The negative suction may in turn facilitate the collection of samples as the suction may cause the samples to be communicated from the scope device into the sample collection jar.

The lower extension member 114 extends from the bottom inner surface 136 of the lid body 102 and is disposed within the interior of the sample collection jar. The lower extension member 114 extends the intake aperture 144 to displace the intake aperture 144 a distance from the bottom inner surface 136 of the lid body 102 and from the suction source aperture 138. The lower extension member 114 may help to prevent samples from being unintentionally communicated into the suction source port 106 by creating a distance between the intake aperture 144 and the suction source aperture 138. As previously described, collected samples within the jar that come within a relatively close proximity to the suction source aperture 138 may be inadvertently suctioned through the suction source aperture 138 and lost into the suction source. The lower extension member 114 causes collected samples to enter the sample collection jar displaced a distance from the suction source aperture 138 and out of relative close proximity to the suction source aperture 138.

The lid body 102 may include a top outer surface 134, a bottom inner surface 136, and a jar collar 148. The lid body 102 may be of any material that is sufficiently rigid to enable the biopsy sample collection device 100 to remain coupled to the sample collection jar. For example, the lid body 102 may be made of polypropylene, other plastics, metal, rubber, or fiberglass. In the embodiment, the lid body 102 is a disk-like shape causing the top outer surface 134 and the bottom inner surface 136 to be circular. As can be appreciated, in other embodiments, the lid body 102, the top outer surface 134, and the bottom inner surface 136 may be some other shape, such as rectangular, square, or elliptical.

The jar collar 148 of the lid body 102 extends orthogonally from the bottom inner surface 136 of the lid body 102 to form a ring to receive a sample collection jar. The jar collar 148 may include screw threads 116 that are configured to cooperate with screw threads on the sample collection jar and receive the sample collection jar. The screw threads 116 may serve to secure the biopsy sample collection device 100 to the sample collection jar. In other embodiments, the screw threads 116 may be of thickness that is greater or less than the thickness of the screw threads 116 shown in the figure. Additionally, in some embodiments the jar collar 148 may include some mechanism other than screw threads to fixedly couple the biopsy sample collection device 100 to the sample collection jar. For example, in some embodiments the jar collar 148 might include a clasp, lock, latch, or clip. In other embodiments, the jar collar 148 may be configured to secure to a sample collection jar with a friction fit. The suction hose clip 110 may be disposed on a lateral edge of the lid body 102. In the illustrated embodiment of FIGS. 2-3, the suction hose clip 110 is disposed on an outer surface of the jar collar 148, which is an outer lateral edge of the lid. The suction hose clip 110 is positioned in line with the suction source port 106 and the scope port 104. The alignment of the suction hose clip 110, the suction source port 106, and the scope port 104 facilitate maintaining the sample collection jar in an upright orientation. The suction hose clip 110 includes two arms disposed in a generally ring-like shape defining a space between the arms into which a suction hose may be inserted. The suction hose clip 110 may be formed of a resilient, flexible material, such that the space between the arms of the suction hose clip 110 may be marginally increased to facilitate the insertion of a suction hose into the clip 110. After insertion of the suction hose, the arms of the suction hose clip 110 may return to their original shape, or otherwise secure or fasten the suction hose into the suction hose clip 110. The suction hose clip 110 may be formed of a sufficiently flexible material such that the arms of the suction hose clip 110 may be marginally extended away from each other and return to their original shape. The material of the suction hose clip 110 may also be sufficiently rigid so as to prevent a suction hose from unintentionally exiting or otherwise disengaging from the suction hose clip 110 during use of the biopsy sample collection device 100.

During a scope procedure using the biopsy sample collection device 100, the suction hose clip 110 creates a coupling between the biopsy sample collection device 100 and the suction hose. The alignment of the suction hose clip 110 with the suction source port 106 and the scope port 104 in turn aligns the engaged suction hose with the suction source port 106 and the scope port 104, which facilitates maintaining the sample collection jar in an upright orientation during manipulation of the scope device for a scope procedure.

In some embodiments, the suction hose clip 110 may be formed of any material that is both sufficiently flexible and rigid to both allow and maintain engagement of a suction hose. For example, the suction hose clip 110 may be formed of polypropylene, other plastics, or a stiff rubber. In other embodiments, the suction hose clip 110 may be of a different size or shape as shown in the figures. In still other embodiments, the biopsy sample collection device 100 may be coupled to a suction hose by a style of clip or fastening mechanism different from the one shown in the figures. For example, the suction hose clip 110 may be replaced by a latch, clasp, or ring configured to engage a suction hose to couple it to the lid body 102 of the biopsy sample collection device 100.

A light pipe clip 108 may be disposed on the lateral edge of the lid body 102, and adjacent to the suction hose clip 110. The light pipe clip 108 may include two arms disposed in a generally ring-like shape and defining a space between the arms into which a light pipe may be inserted. The light pipe clip 108 may be formed of a resilient, flexible material, such that the space between the arms of the light pipe clip 108 may be marginally increased to facilitate the insertion of a light pipe into the light pipe clip 108. After insertion of the light pipe, the arms of the light pipe clip 108 may return to their original shape, or otherwise secure or fasten the light pipe to the light pipe clip 108. The light pipe clip 108 may be formed of a sufficiently flexible material such that the arms of the clip may be marginally extended away from each other, then later return to their original shape. The material of the light pipe clip 108 may also be sufficiently rigid as to prevent a light pipe from unintentionally exiting or otherwise disengaging from the light pipe clip 108 during use of the biopsy sample collection device 100. For example, the light pipe clip 108 may be formed of polypropylene, other plastics, or rubber. The light pipe clip 108 may be formed of the same material as the suction hose clip 110.

In some embodiments, the light pipe clip 108 may be of a different size or shape as shown in the figures. In other embodiments, the biopsy sample collection device 100 may be coupled to a light pipe by a clip or fastening mechanism different from the one shown in the figures. For example, the light pipe clip 108 may be replaced by a latch, clasp, or ring.

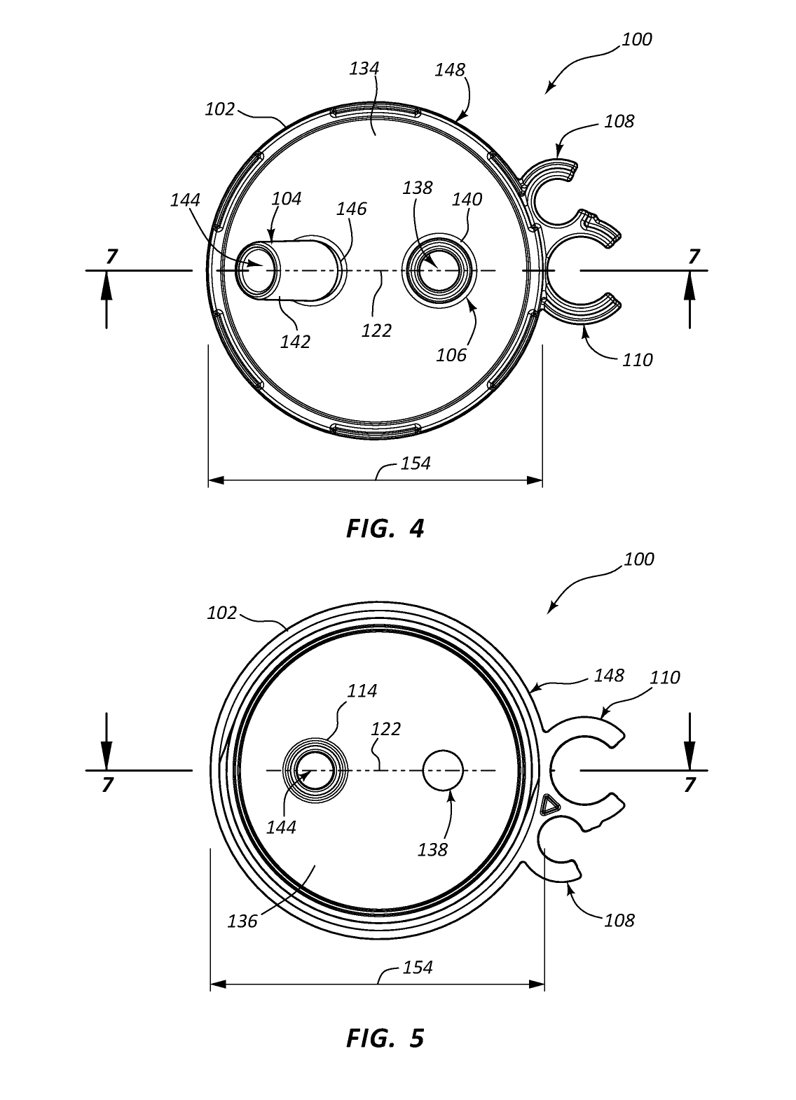

FIGS. 4 and 5 are respectively a top and bottom view of the biopsy sample collection device 100 of FIG. 1. Referring generally and collectively to FIGS. 4 and 5, the illustrated biopsy sample collection device 100 includes the lid body 102, the suction source port 106, the scope port 104, the suction hose clip 110, and the light pipe clip 108.

The suction source port 106, the scope port 104, and the suction hose clip 110 are shown aligned along a line 122 which intersects a center of the circularly shaped top outer surface 134 of the biopsy sample collection device 100. Stated otherwise, the suction source port 106, the scope port 104, and the suction hose clip 110 are shown in alignment along a line 122 that is a diameter 154 of the lid body 102 through a center of the top outer surface 134. In the illustrated embodiment, the line 122 is a centerline of the lid body 102. In other embodiments of the biopsy sample collection device 100, the suction source port 106, the scope port 104, and the suction hose clip 110 may all be disposed along another line of alignment that may not intersect the center of the biopsy sample collection device 100. The distance between this other line of alignment and the line 122 shown in the figure may be a fraction of the diameter 154 of the device 100. In still other embodiments, the suction source port 106, the scope port 104, and the suction hose clip 110 may be disposed such that no significant alignment exists between these components, or the ports 104, 106 may be disposed in such a way as to form an alignment with one another without forming an alignment with the suction hose clip 110. In yet another embodiment, one of the ports 104, 106 may form an alignment with the suction hose clip 110 without forming an alignment with the other port 104, 106.

The suction source port 106 includes the connection member 140 and the suction source aperture 138 through the lid body 102. The suction source port 106 is disposed on and extends from the upper surface 134 of the lid body 102 on the line 122, in line with the scope port 104 and the suction hose clip 110. The suction source aperture 138 provides fluid communication from a suction source to an interior of the sample collection jar. The connection member 140 may couple to a suction source to transfer suction from the suction source through the suction source aperture 138 and create a negative pressure in the sample collection jar. In the embodiment, the connection member 140 of the suction source port 106 extends away from and substantially orthogonal to the top outer surface 134 of the lid body 102.

The suction source aperture 138, in the illustrated embodiment of FIG. 5, is disposed in the lid body 102 substantially flush with the bottom inner surface 136 of the lid body 102. As can be appreciated, in other embodiments, the suction source aperture 138 may be extended to be displaced from or otherwise not flush with the bottom inner surface 136.

The scope port 104 of the biopsy sample collection device 100 includes the intake aperture 144, the upper extension member 146, and the angled connection member 142. The intake aperture or intake lumen 144 may be defined through the lid body 102 and extend as a lumen through the scope port 104. The intake aperture 144 may serve to provide fluid communication to the interior of the sample collection jar and to transfer negative pressure within the sample collection jar to a scope device. The angled connection member 142 extends from the upper extension member 146 at an angle away from and in line with the suction source port 106. In other words, the angled connection member 142 angles away from the upper extension member 146 in a direction along the line 122. The angled connection member 142 may facilitate coupling the scope port 104 to a collection lumen of a scope device and to transfer suction from the interior of the sample collection jar through the intake aperture 144, creating negative pressure within the collection lumen of the scope device and enabling the collection of a biopsy sample. The angled connection member 142 facilitates maintaining a sample collection jar in an upright position or orientation during manipulation of the scope device during a scope procedure.

The lid body 102, as shown in FIGS. 4 and 5, includes the top outer surface 134, the bottom inner surface 136, and the jar collar 148. In the illustrated embodiment, the lid body 102 is a disk-like shape causing the top outer surface 134 to be circular. As can be appreciated, in other embodiments the lid body 102 and the top outer surface 134 may be rectangular or square in shape. The jar collar 148 is configured to receive and/or engage the sample collection jar. The lid body 102 may be formed of any sufficiently rigid material such as plastic, metal, or fiberglass.

The suction hose clip 110, as shown in FIGS. 4 and 5, is disposed on the lateral of the lid body 102 in line with the suction source port 106 and the scope port 104. The suction source port 106, the scope port 104, and the suction hose clip 110 are aligned along a line 122 that is a diameter 154 of the lid body 102 through a center of the lid body 102.

The light pipe clip 108 is disposed on the lateral edge adjacent to the suction hose clip 110. The biopsy sample collection device 100 of the figures includes the light pipe clip 108 on a given side of the suction hose clip 110. However, as can be appreciated, in other embodiments the light pipe clip 108 may be disposed on an opposite adjacent side of the suction hose clip 110.

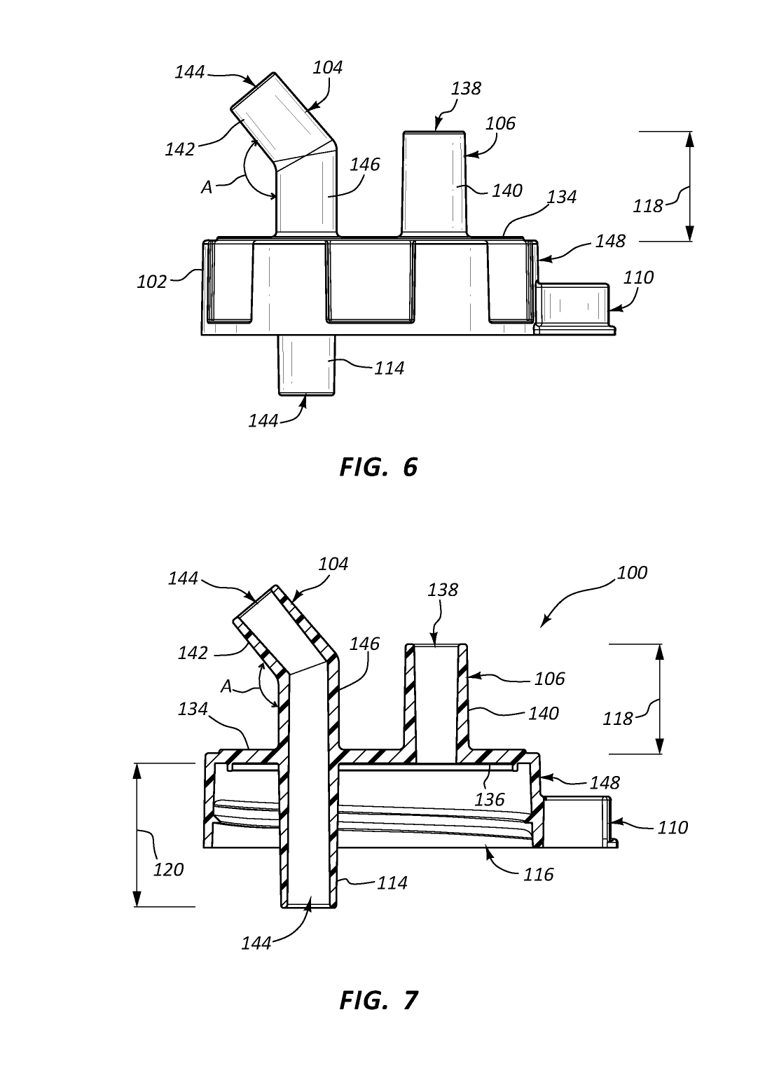

FIG. 6 is a side view and of the biopsy sample collection device 100 of FIG. 1. FIG. 7 is a side cross sectional view of the biopsy sample collection device 100, taken along the line 7-7 of FIGS. 4 and 5. Referring to FIGS. 6 and 7, generally and collectively, the illustrated biopsy sample collection device 100 includes the suction source port 106, the scope port 104, the lid body 102, the suction hose clip 110, as described above.

The suction source port 106 includes the connection member 140 and the suction source aperture 138 through the lid body 102. FIGS. 6 and 7 illustrate that the suction source port 106, and more specifically the connection member 140, is disposed on and extends from the top outer surface 134 of the lid body 102. The connection member 140 extends from the lid body 102 in a direction parallel to a longitudinal axis of the sample collection jar. The suction source aperture 138 is elongated by and extends as a lumen through the connection member 140 of the suction source port 106. The connection member 140 orthogonally extends a distance 118 away from the top outer surface 134 of the lid body 102. The suction source aperture 138 is also disposed at, or substantially flush with, the bottom inner surface 136 of the lid body 102

The scope port 104 of the biopsy sample collection device 100 may include an intake aperture 144, an upper extension member 146, an angled connection member 142, and a lower extension member 114. The intake aperture 144 may be defined through the lid body 102 by the scope port 104, extending as a lumen through the angled connection member 142, the upper extension member 146, and the lower extension member 114.

As shown in FIGS. 6 and 7, the angled connection member 142 extends from the upper extension member 146 and at an angle A. The angled connection member 142 angles at the angle A from the upper extension member 146 in a direction away from and in line with the suction source port 106. The angled connection member 142 also is disposed at the angle A relative to a longitudinal axis of the sample collection jar. In some embodiments, the angle A may be between 110 degrees and 150 degrees. In some embodiments, the angle A may be between 120 degrees and 140 degrees. In some embodiments, the angle A may be between 125 degrees and 135 degrees. In certain embodiments, the angle A may be 130 degrees.

The lower extension member 114 extends orthogonally from the bottom inner surface 136 of the lid body 102 to be disposed within the interior of the sample collection jar. The lower extension member 114 displaces the intake aperture 144 a distance 120 from the bottom inner surface 136 of the lid body 102 and from the suction source aperture 138. The lower extension member 114 and the resulting distance 120 between the intake aperture 144 and the suction source aperture 138 may help to limit samples from being unintentionally communicated into the suction source aperture 138 and lost to the suction source through the suction source port 106. The suction within the interior of the sample jar may be greatest or strongest adjacent to the suction source aperture 138. Accordingly, displacing the intake aperture 144 a distance from the suction source aperture 138 (and/or a distance from the bottom inner surface 136 of the lid body 102) can ensure that collected biopsy samples enter the collection jar safely out of suction range of the suction source aperture 138. Maintaining the collection jar in an upright configuration can also help ensure that collected biopsy samples are not lost to suction through the suction source aperture 138. In other embodiments of the biopsy sample collection device 100, the distance 120 may be greater or less than the distance 120 shown in the figure. In yet other embodiments, the lower extension member 114 may be coupled to or operate in conjunction with a sample trap or basket, disposed within the interior of the sample collection jar. The trap or basket can strain or otherwise collect solid tissue biopsy samples from fluid biopsy samples.

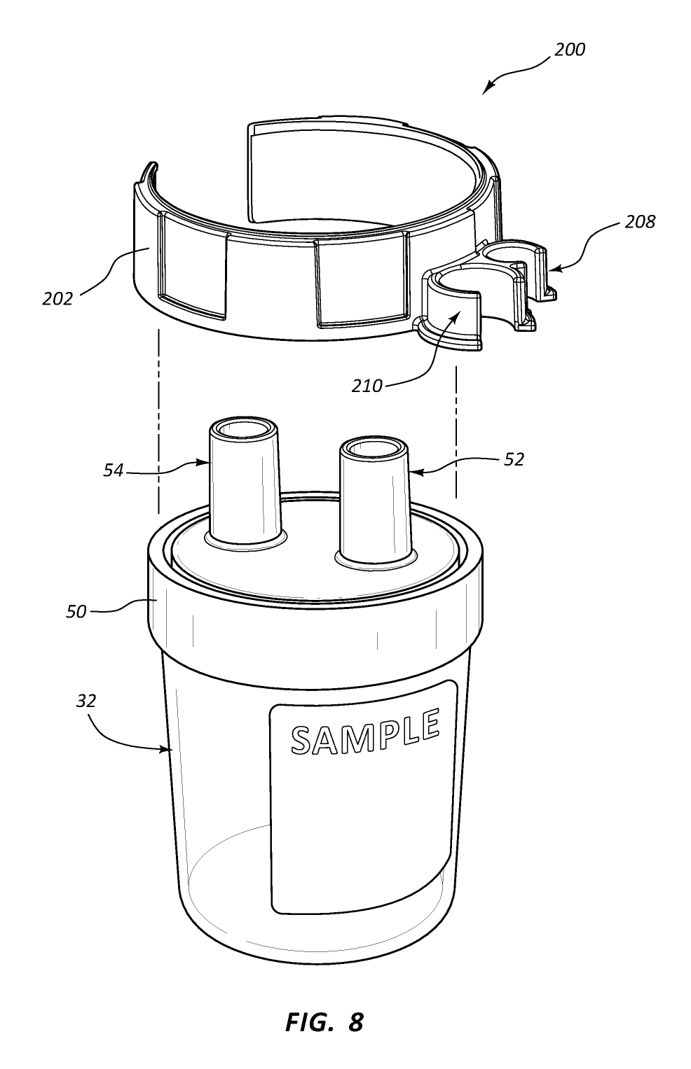

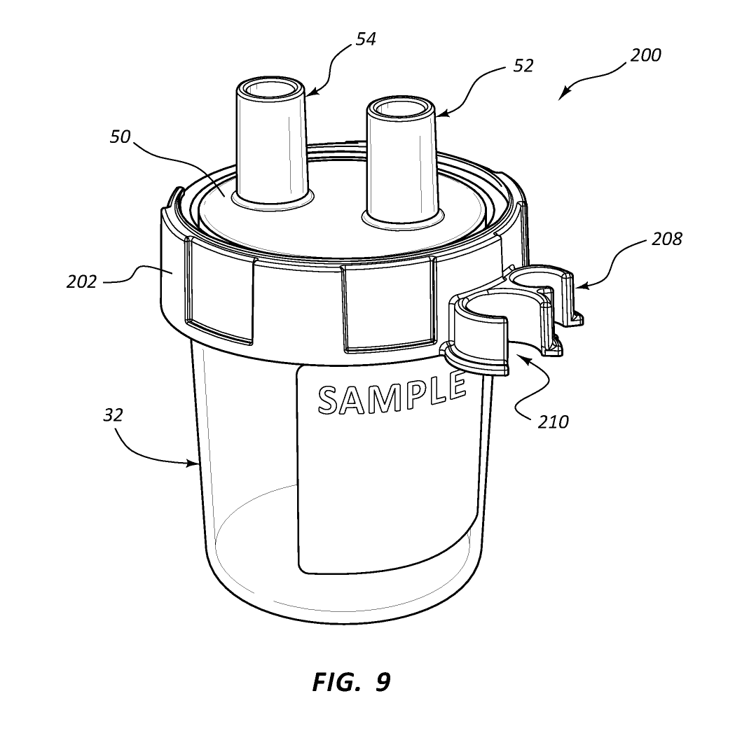

FIG. 8 is a perspective view of a biopsy sample collection device 200, according to another embodiment of the present disclosure, uncoupled from a sample collection jar lid 50. FIG. 9 is a perspective view of the biopsy sample collection device of FIG. 8 coupled to the biopsy collection jar lid 50. Referring generally and collectively to FIGS. 8 and 9, the illustrated biopsy sample collection device 200 includes a ring body 202, a suction hose clip 210, and a light pipe clip 208.

The ring body 202 is ring-like in shape and is configured to couple to a traditional lid 50 of the sample collection jar 32. A practitioner may position the ring body 202 over and into engagement with the lid 50 by sliding the ring body 202 over the lid 50. In other embodiments, the arms of the ring body 202 may be marginally expanded or extended to facilitate insertion of the lid 50 into the interior of the ring body 202. In still other embodiments, the ring body 202 may be of a different shape or size than shown, and/or may include a mechanism that may allow the ring body 202 to be fastened to the lid 50. The ring body 202 may be formed of any sufficiently rigid material to be secured to the lid 50.

The suction hose clip 210 may be disposed on an outer lateral edge of the ring body 202. Further, the suction hose clip 210 can be positioned to be along a diameter of the lid 50, in line with the ports 52, 54 of the lid 50. The suction hose clip 210 may include two arms disposed in a generally ring-like shape with a space between the arms, into which a suction hose may be inserted. The space between the arms of the suction hose clip 210 may be marginally increased, which may facilitate the insertion of a suction hose into the clip 210. After insertion of the suction hose, the arms of the clip 210 may return to their original shape or otherwise secure or fasten the suction hose to the clip 210. The suction hose clip 210 may be of any sufficiently flexible material such that the arms of the clip 210 may be marginally extended away from each other and return to their original shape. The material of the suction hose clip 210 may also be sufficiently rigid to prevent a suction hose from unintentionally exiting the clip 210 during operation of the device 200. The clip 210 may facilitate maintaining an upright orientation of the biopsy sample collection device 200 during use of the biopsy sample collection device 200 with a scope device during a scope procedure. Aligning the suction hose clip 210 and the ports 52, 54 can facilitate maintaining the sample collection jar 32 in an upright orientation. In other embodiments, the suction hose clip 210 may be of a different size, shape, or material that may be suitable for facilitating the coupling of the suction hose clip 210 to a corresponding suction hose. In yet other embodiments, the biopsy sample collection device 200 may be coupled to a suction hose by some style of clip or fastening mechanism different from the one shown in the figures. For example, the clip 210 may be replaced by a latch, clasp, or ring.

The light pipe clip 208 may be disposed on the outer lateral edge of the ring body 202, and adjacent to the suction hose clip 210. The light pipe clip 208 may include two arms disposed in a generally ring-like shape with a space between the arms into which a light pipe may be inserted into the clip 208. The light pipe clip 208 may be formed of a resilient, flexible material, such that the space between the arms of the light pipe clip 208 may be marginally increased, which may facilitate the insertion of a light pipe into the light pipe clip 208. After insertion of the light pipe, the arms of the light pipe clip 208 may return to their original shape or otherwise secure or fasten the light pipe to the clip 208. The light pipe clip 208 may be of a sufficiently flexible material such that the arms of the clip may be marginally extended away from each other, and then later return to their original shape. The material of the light pipe clip 208 may also be sufficiently rigid to prevent a light pipe from unintentionally exiting the light pipe clip 208. The light pipe clip 208 may be of any material that is both sufficiently rigid and flexible, such as polypropylene, other plastics, or rubber. In other embodiments, the light pipe clip 208 may be of a different size, shape, or material that may be suitable for facilitating the coupling of the light pipe clip 208 to a corresponding light pipe. In still other embodiments, the biopsy sample collection device 200 may be coupled to a light pipe by some style of clip or fastening mechanism different from the one shown in the figures. For example, the light pipe clip 208 may be replaced by a latch, clasp, or ring.

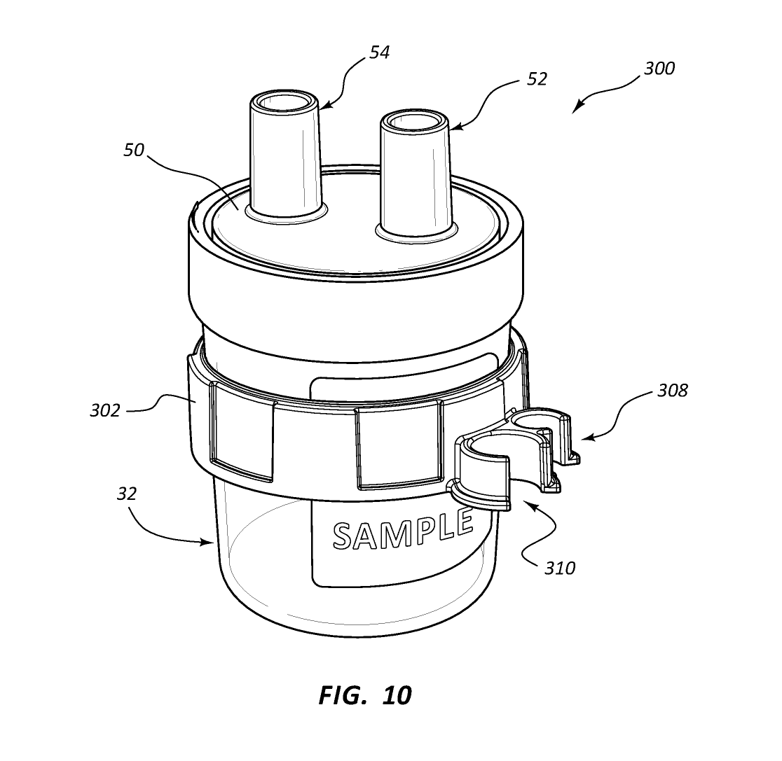

FIG. 10 is a perspective view of a biopsy sample collection device 300, according to still another embodiment, coupled to the sample collection jar 32. The biopsy sample collection device 300 includes a ring body 302, a suction hose clip 310, and a light pipe clip 308. The ring body 302 is ring-like in shape and is configured to couple to and/or engage the sample collection jar 32. Engagement with the sample collection jar 32 may be on sidewalls of the sample collection jar 32, below a lid 50. A practitioner may position the ring body 302 around and into engagement with the sample collection jar 32 by sliding the ring body 302 around a bottom of the sample collection jar 32 and up the sidewalls to a friction fit engagement around the sample collection jar 32. In other embodiments, the ring body 302 may be of a different shape or size than shown, and/or may include a mechanism that may allow the ring body 302 to be fastened to the sample collection jar 32. The ring body 302 may be formed of any sufficiently rigid material to be secured to around the sample collection jar 32.

The suction hose clip 310 may be disposed on an outer lateral edge of the ring body 302. Further, the suction hose clip 310 can be positioned in line with the ports 52, 54 of the lid 50 of the sample collection jar 32.

The light pipe clip 308 is disposed on the lateral edge adjacent to the suction hose clip 310. The biopsy sample collection device 300 includes the light pipe clip 308 on a given side of the suction hose clip 310. However, as can be appreciated, in other embodiments the light pipe clip 308 may be disposed on an opposite adjacent side of the suction hose clip 310.

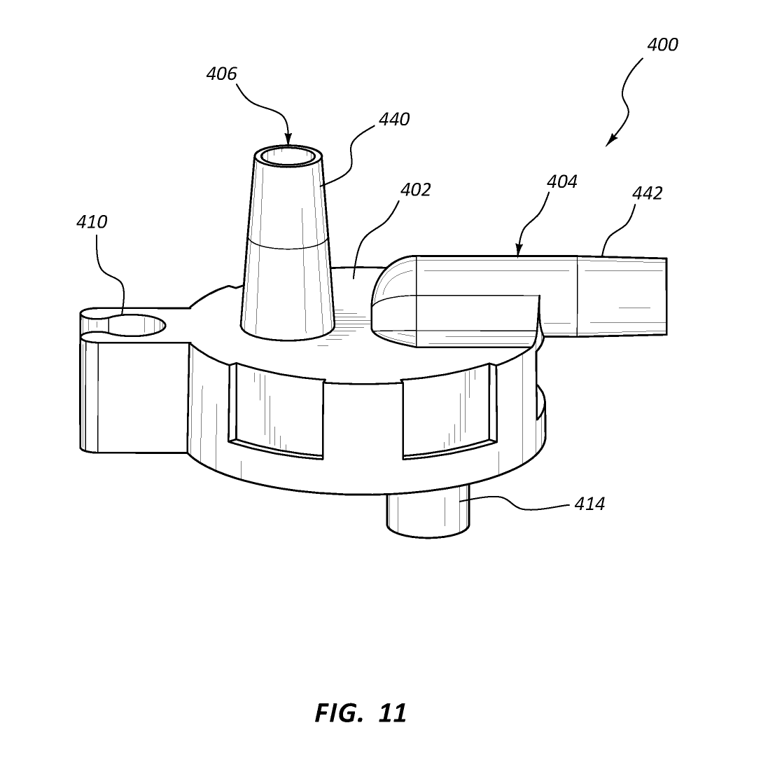

FIG. 11 is a side perspective view of a biopsy sample collection device 400, according to still another embodiment. The biopsy sample collection device 400 includes a lid body 402, a scope port 404 and a suction source port 406, and a suction hose clip 410. The scope port 404 and the suction source port 406 are disposed on the lid body 402.

The suction source port 406 includes a connection member 440 and provides a suction source aperture defined in and through the lid body 402. The suction source aperture provides fluid communication from a suction source to an interior of a sample collection jar (e.g., see the jar 32 of FIG. 1) to which the biopsy sample collection device 400 may be secured. The connection member 440 may be configured to couple to a suction source to facilitate transfer of suction from a suction source through the suction source aperture. More specifically, the connection member 440 is configured to receive or otherwise couple to a suction hose 28 (see FIG. 1), which is coupled to the suction source. The suction hose 28 may couple to the connection member 440, for example, with a friction fit. The transfer of suction through the suction source aperture may then create a negative pressure in the interior of the sample collection jar. The negative pressure within the jar may aid in communicating samples from a scope device to the jar, as described above. In the embodiment of FIG. 11, the connection member 440 of the suction source port 406 extends away from and substantially orthogonal to a top outer surface of the lid body 402.

In other embodiments, the suction source port 406 may couple to a suction source in a variety of different ways. For example, the suction source port 406 may be configured to couple to a suction source with a threaded socket, a lock, a clamp, a latch, or other fastening mechanism. The suction source port 406 may include one or more of these examples in conjunction with the connection member 440 or in place of the connection member 440.

The scope port 404 of the biopsy sample collection device 400 includes an angled connection member 442 and a lower extension member 414 and provides an intake aperture through the lid body 402. The intake aperture may be extended or lengthened as a lumen through the scope port 404 (e.g., through the lower extension member 414 and the angled connection member 442). The intake aperture provides fluid communication to the interior of the sample collection jar, to transfer negative pressure from within the sample collection jar to a scope device, and to receive samples from a scope device. The transfer of negative pressure from the interior of the sample collection jar to a scope device may aid to communicate samples from a scope device to pass through the intake aperture and enter the sample collection jar.

In the biopsy sample collection device 400 of FIG. 11, the angled connection member 442 extends at an angle away from and in line with the suction source port 406. For example, the angled connection member 442 may be disposed on and extend in a direction parallel to the top outer surface of the lid body 402, which is at an angle that is substantially orthogonal or perpendicular to the suction source port 406. In other embodiments, the angled connection member 442 may be oriented at an angle between 0 degrees and 60 degrees relative to the top outer surface of the lid body 402 (e.g., at an angle between 30 degrees and 90 degrees relative to the suction source port 406). The angled connection member 442 facilitates coupling the scope port 404 to a collection lumen of a scope device. More specifically, the angled connection member 442 is configured to receive or otherwise couple to a sample collection tube 24 (see FIG. 1), which is coupled to the scope device. The sample collection tube 24 may be Tygon® tubing, silicone tubing, or other appropriately flexible yet sufficiently rigid material. The angled connection member 442 facilitates maintaining the biopsy sample collection device 400 in an upright orientation. The angled connection member 442 also facilitates transfer of suction from the interior of the sample collection jar through the intake aperture. The transfer of suction may then create a negative pressure within the collection lumen of the scope device. The negative suction may in turn facilitate the collection of samples as the suction may cause the samples to be communicated from the scope device into the sample collection jar.

The examples and embodiments disclosed herein are to be construed as merely illustrative and exemplary, and not a limitation of the scope of the present disclosure in any way.

It will be obvious to those having skill in the art that many changes may be made to the details of the above-described embodiments without departing from the underlying principles of the invention. The scope of the present invention should, therefore, be determined only by the following claims.

We claim:

1. A biological sample collection device comprising:
a lid body including a top outer surface and a bottom inner surface configured to couple to a sample collection jar;
a suction source port disposed on the lid body, the suction source port defining a suction source aperture through the lid body to provide fluid communication to an interior of the sample collection jar, the suction source port including a connection member to couple to a suction hose from a suction source to transfer suction through the source aperture to create negative pressure within the sample collection jar; and
a scope port disposed in the lid body, the scope port defining an intake aperture through the lid body to provide fluid communication to the interior of the sample collection jar to transfer negative pressure within the sample collection jar to a scope device to receive samples from the scope device, wherein the scope port includes:
an upper extension member extending from the outer surface of the lid body, the upper extension in line with the suction source port; and
an angled connection member extending from the upper extension member at an angle away from and in line with the suction source port, the angled connection member to couple to a sample collection tube in fluid communication with a collection lumen of the scope device to transfer suction from the interior of the jar through the intake aperture and create negative pressure within a collection lumen of the scope device for collecting samples.

2. The biological sample collection device of claim 1, wherein the connection member of the suction source port is disposed on the outer surface of the lid body and extends away from the outer surface of the lid body.

3. The biological sample collection device of claim 1, wherein the scope port further comprises a lower extension member that extends from the inner surface of the lid body to be disposed within the interior of the sample collection jar, the lower extension member displacing the intake aperture a distance from the inner surface of the lid body and from the source aperture.

4. The biological sample collection device of claim 1, further comprising a suction hose clip disposed on a lateral edge of the lid body and in line with both the suction source port and the scope port, the suction hose clip configured to engage a suction hose coupled to the suction source port.

5. The biological sample collection device of claim 4, further comprising a light pipe clip disposed on the lateral edge of the lid body adjacent the suction hose clip, the light pipe clip configured to engage a light pipe to secure the biological sample collection device relative to the light pipe.

6. The biological sample collection device claim 1, wherein the suction source port and the scope port are aligned on a diameter of the lid body.

7. The biological sample collection device of claim 1, wherein the angled connection member is configured to position the biological sample collection device to maintain the sample collection jar in an upright orientation while in use with and coupled to a scope device during a scope biopsy procedure.

8. The biological sample collection device of claim 1, further comprising a sample collection jar configured to couple to the lid body.

9. A biological sample collection device comprising:
a ring body configured to engage a sample collection jar that includes a lid comprising a suction source port and a scope port, the suction source port including a connection member extending away from an outer surface of the lid, the scope port including an upper extension member extending away from and substantially orthogonal to the outer surface of the lid;
a suction hose clip disposed on a lateral edge of the ring body to be positioned in line with both the suction source port and the scope port, the suction hose clip configured to engage a suction hose coupled to the suction source port to secure the biological sample collection device relative to the suction hose to maintain the sample collection jar in an upright orientation.

10. The biological sample collection device of claim 9, further comprising a light pipe clip disposed on the lateral edge of the ring body and adjacent the suction hose clip.

11. The biological sample collection device of claim 9, wherein the suction hose clip is configured to align with the suction source port and the scope port on a diameter of the main body.

12. The biological sample collection device of claim 9, wherein the ring body is configured to engage the lid of the sample collection jar.

13. The biological sample collection device of claim 9, wherein the ring body is configured to engage a sidewall of the sample collection jar, below the lid of the sample collection jar.

14. The biological sample collection device of claim 9, wherein the suction source port and the scope port are aligned on a centerline of the lid of the sample collection jar.

15. The biological sample collection device of claim 9, wherein the scope port transfers negative pressure within the sample collection jar to a scope device that is an endoscope used for visual examination of an interior of a body cavity.

16. A method for collecting a biological sample, comprising:
coupling a biological sample collection device to a sample collection jar, the biological sample collection device including a scope port and a suction port, the scope port and the suction port providing fluid communication to an interior of the sample collection jar;
coupling a sample collection tube to an endoscope and the scope port, such that the sample collection tube is coupled to the scope port at an angle offset from a longitudinal axis of the sample collection jar, wherein the angle positions the biological sample collection device to maintain the sample collection jar at a substantially upright orientation;
coupling a suction hose to a suction source and to the suction port;
applying a negative pressure from the vacuum source to the interior of the sample collection jar; and
collecting a biological sample from the endoscope into the sample collection jar.

17. The method of claim 16, wherein the biological sample collection device further includes a suction hose clip, the method further comprising:
coupling the second hose to the suction hose clip such that a loop is formed above the biological sample collection device to maintain the sample collection jar in an upright position.

18. The method of claim 16, wherein the biological sample collection device further includes a light pipe clip, the method further comprising:
coupling a light pipe of the endoscope to the light pipe clip to secure the sample collection relative to the light pipe.

19. A biological sample collection device comprising:
a lid body including a top outer surface and a bottom inner surface configured to couple to a sample collection jar;
a suction source port disposed on the lid body, the suction source port defining a suction source aperture through the lid body to provide fluid communication to an interior of the sample collection jar, the suction source port including a connection member to couple to a suction hose from a suction source to transfer suction through the source aperture to create negative pressure within the sample collection jar; and
a scope port disposed in the lid body, the scope port defining an intake aperture through the lid body to provide fluid communication to the interior of the sample collection jar to transfer negative pressure within the sample collection jar to a scope device to receive samples from the scope device, wherein the scope port comprises an angled connection member extending at an angle away from and in line with the suction source port, the angled connection member to couple to a sample collection tube in fluid communication with a collection lumen of the scope device to transfer suction from the interior of the jar through the intake aperture and create negative pressure within a collection lumen of the scope device for collecting samples.

20. The biological sample collection device of claim 19, wherein the angled connection member of the scope port extends away from the suction source port parallel to a top outer surface of the lid body.

21. The biological sample collection device of claim 19, wherein the angled connection member of the scope port extends away from the suction source port at an angle between 30 degrees and 90 degrees.
